(19) United States
(12) Patent Application Publication
(10) Pub. No.: US 2026/0182953 A1
(43) Pub. Date: Jul. 2, 2026

(54) ULTRASOUND DIAGNOSTIC APPARATUS AND CONTROL METHOD OF ULTRASOUND DIAGNOSTIC APPARATUS

(71) Applicant: FUJIFILM Corporation, Tokyo (JP)

(72) Inventor: Tsuyoshi MATSUMOTO, Kanagawa (JP)

(73) Assignee: FUJIFILM Corporation, Tokyo (JP)

(21) Appl. No.: 19/396,359

(22) Filed: Nov. 21, 2025

(30) Foreign Application Priority Data
Dec. 26, 2024 (JP) 2024-229886

(51) Int. Cl. A61B 8/08; A61B 8/00

(52) U.S. Cl. CPC A61B 8/0891; A61B 8/4254; A61B 8/461; A61B 8/483

(57) ABSTRACT

Provided are an ultrasound diagnostic apparatus and a control method of the ultrasound diagnostic apparatus that make it easy to acquire an ultrasound image of a blood vessel region suitable for interion of an insertion object.

An ultrasound diagnostic apparatus includes: a position sensor that acquires position information of an ultrasound probe; an image acquisition unit that acquires a plurality of frames of ultrasound images by transmitting and receiving ultrasound beams using the ultrasound probe; a three-dimensional image data generation unit that generates three-dimensional ultrasound image data based on the position information of the ultrasound probe and the plurality of frames of ultrasound images representing a short-axis image of the blood vessel; a centerline acquisition unit that acquires a centerline of the blood vessel in a three-dimensional space based on the three-dimensional ultrasound image data; a meandering degree calculation unit that calculates a meandering degree of the centerline in a transverse diameter direction of the blood vessel; and a guide unit that guides the ultrasound probe to a range on the centerline based on the meandering degree.

### CROSS-REFERENCE TO RELATED APPLICATIONS

The present application claims priority under 35 U.S.C. § 119 to Japanese Patent Application No. 2024-229886, filed on December 26, 2024. The above application is hereby expressly incorporated by reference, in its entirety, into the present application.

### BACKGROUND OF THE INVENTION

### 1. Field of the Invention

The present invention relates to an ultrasound diagnostic apparatus used for observing a blood vessel in a subject in which puncture is performed and a control method of the ultrasound diagnostic apparatus.

### 2. Description of the Related Art

In the related art, a procedure of puncturing a blood vessel with a so-called puncture needle or the like while observing a blood vessel in a subject using a so-called ultrasound diagnostic apparatus has been known. In such a procedure, by checking a plurality of frames of ultrasound images captured along a longitudinal direction of the blood vessel in real time, a blood vessel region suitable for puncture is often searched for, such as a blood vessel that does not meander in a direction orthogonal to the longitudinal direction of the blood vessel and parallel to a body surface of the subject. In this case, a user of the ultrasound diagnostic apparatus, such as a doctor, usually searches for a blood vessel region suitable for puncture by alternately capturing a blood vessel short-axis image representing a blood vessel cross section orthogonal to the longitudinal direction of the blood vessel and a blood vessel long-axis image representing a blood vessel longitudinal section along the longitudinal direction of the blood vessel.

The procedure of searching for a blood vessel region suitable for puncture by checking the ultrasound image requires repeatedly capturing and checking the short-axis image and the long-axis image of the blood vessel, which can require a great deal of effort on the part of the user to specify the blood vessel region suitable for puncture. Therefore, for example, as disclosed in JP2017-018195A, it is considered to generate a three-dimensional ultrasound image of a blood vessel from a plurality of two-dimensional frames of ultrasound images obtained by capturing the blood vessel, check the generated three-dimensional ultrasound image, and specify a blood vessel region suitable for puncture.

### SUMMARY OF THE INVENTION

However, although the technique of JP2017-018195A can specify an approximate position of the blood vessel region suitable for puncture, in order to accurately capture an ultrasound image of the blood vessel region suitable for puncture, the user needs to determine an accurate position of the blood vessel region suitable for puncture by capturing a plurality of frames of ultrasound images representing the short-axis image and the long-axis image of the blood vessel in the vicinity of the specified approximate position, which may require a large amount of effort on the part of the user.

The present invention has been made to solve such a problem in the related art, and an object of the present invention is to provide an ultrasound diagnostic apparatus and a control method of the ultrasound diagnostic apparatus that make it easy to acquire an ultrasound image of a blood vessel region suitable for insertion of an insertion object.

According to the following configuration, the above-described object can be achieved.

[1] An ultrasound diagnostic apparatus comprising:

an ultrasound probe;

a position sensor that acquires position information of the ultrasound probe;

an image acquisition unit that acquires a plurality of frames of ultrasound images obtained by capturing a blood vessel of a subject by transmitting and receiving ultrasound beams using the ultrasound probe, the plurality of frames of ultrasound images representing a short-axis image of the blood vessel;

a three-dimensional image data generation unit that generates three-dimensional ultrasound image data of the subject based on the position information of the ultrasound probe acquired by the position sensor and the plurality of frames of ultrasound images acquired by the image acquisition unit;

a centerline acquisition unit that acquires a centerline of the blood vessel in a three-dimensional space based on the three-dimensional ultrasound image data generated by the three-dimensional image data generation unit;

a meandering degree calculation unit that calculates a meandering degree of the centerline acquired by the centerline acquisition unit in a transverse diameter direction of the blood vessel, the transverse diameter direction being perpendicular to a plane corresponding to the short-axis image; and

a guide unit that guides the ultrasound probe to a range on the centerline based on the meandering degree calculated by the meandering degree calculation unit.

[2] The ultrasound diagnostic apparatus according to [1],

in which the meandering degree calculation unit

divides the centerline into a plurality of sections having a predetermined length, and

calculates the meandering degree in each of the plurality of sections.

[3] The ultrasound diagnostic apparatus according to [2],

in which the meandering degree calculation unit

calculates an average position of the centerline in the transverse diameter direction, and

calculates, in each of the plurality of sections, the number of inflection points of the centerline whose distance from the average position is equal to or greater than a predetermined position threshold value, as the meandering degree.

[4] The ultrasound diagnostic apparatus according to [2],

in which the meandering degree calculation unit calculates, in the plurality of sections, a reciprocal of an interval between adjacent inflection points of the centerline, as the meandering degree.

[5] The ultrasound diagnostic apparatus according to any one of [1] to [4],

in which the guide unit guides the ultrasound probe to a range on the centerline where the meandering degree calculated by the meandering degree calculation unit is equal to or less than a predetermined meandering degree threshold value.

[6] The ultrasound diagnostic apparatus according to any one of [1] to [4],

in which the guide unit

acquires a depth of the blood vessel with respect to a body surface of the subject over an entire centerline by referring to the three-dimensional ultrasound image data generated by the three-dimensional image data generation unit, and

guides the ultrasound probe to a range where the meandering degree is equal to or less than a predetermined meandering degree threshold value and the depth of the blood vessel is equal to or less than a predetermined depth threshold value.

[7] The ultrasound diagnostic apparatus according to any one of [1] to [4],

in which the guide unit

acquires an inner diameter of the blood vessel over an entire centerline by referring to the three-dimensional ultrasound image data generated by the three-dimensional image data generation unit, and

guides the ultrasound probe to a range where the meandering degree is equal to or less than a predetermined meandering degree threshold value and the inner diameter of the blood vessel is closest to a predetermined recommended inner diameter value.

[8] The ultrasound diagnostic apparatus according to [2] or [3],

in which the meandering degree is equal to or less than a predetermined meandering degree threshold value in two or more of the plurality of sections, and

the guide unit guides the ultrasound probe to a section having a smallest meandering degree among the two or more sections.

[9] The ultrasound diagnostic apparatus according to any one of [1] to [8], further comprising:

a monitor,

in which the guide unit displays a guide for the ultrasound probe on the monitor.

The ultrasound diagnostic apparatus according to [9],

in which a marker is disposed on the ultrasound probe,

the position sensor includes

an optical camera that acquires an optical image in which the ultrasound probe is captured, and

a marker detection unit that acquires the position information of the ultrasound probe by detecting the marker captured in the optical image acquired by the optical camera, and

the guide unit displays the guide for the ultrasound probe on the monitor by superimposing the guide for the ultrasound probe on the optical image acquired by the optical camera based on the position information of the ultrasound probe acquired by the marker detection unit.

The ultrasound diagnostic apparatus according to any one of [1] to [10], further comprising:

an optical camera that acquires an optical image in which the ultrasound probe and a specific part of the subject are captured; and

a relative position information conversion unit that converts, based on the position information acquired by the position sensor and the optical image acquired by the optical camera, the position information into relative position information with respect to the specific part captured in the optical image,

in which the three-dimensional image data generation unit uses the relative position information converted by the relative position information conversion unit as the position information of the ultrasound probe acquired by the position sensor.

The ultrasound diagnostic apparatus according to any one of [1] to [11],

in which a plurality of the blood vessels are captured in each of the plurality of frames of ultrasound images,

the ultrasound diagnostic apparatus further comprises an attention degree calculation unit that calculates an attention degree of each of the plurality of blood vessels based on positions of the plurality of blood vessels in each of the plurality of frames of ultrasound images or a length of the centerline acquired by the centerline acquisition unit for each of the plurality of blood vessels, and

the guide unit guides the ultrasound probe on a blood vessel having a largest attention degree among a plurality of the attention degrees calculated by the attention degree calculation unit, based on the meandering degree calculated by the meandering degree calculation unit.

The ultrasound diagnostic apparatus according to any one of [1] to [11],

in which a plurality of the blood vessels are captured in each of the plurality of frames of ultrasound images,

the ultrasound diagnostic apparatus further comprises a suitability degree calculation unit that calculates a suitability degree of each of the plurality of blood vessels based on a depth of the blood vessel with respect to a body surface of the subject or an inner diameter of the blood vessel by referring to the three-dimensional ultrasound image data generated by the three-dimensional image data generation unit, and

the guide unit guides the ultrasound probe on a blood vessel having a largest suitability degree among a plurality of the suitability degrees calculated by the suitability degree calculation unit, based on the meandering degree calculated by the meandering degree calculation unit.

A control method of an ultrasound diagnostic apparatus, the control method comprising:

acquiring position information of an ultrasound probe;

acquiring a plurality of frames of ultrasound images that are obtained by capturing a blood vessel of a subject by transmitting and receiving ultrasound beams using the ultrasound probe and that represent a short-axis image of the blood vessel;

generating three-dimensional ultrasound image data of the subject based on the position information of the ultrasound probe and the plurality of frames of ultrasound images;

acquiring a centerline of the blood vessel in a three-dimensional space based on the three-dimensional ultrasound image data;

calculating a meandering degree of the centerline in a transverse diameter direction of the blood vessel, the transverse diameter direction being perpendicular to a plane corresponding to the short-axis image of the blood vessel; and

guiding the ultrasound probe to a range on the centerline based on the meandering degree.

An ultrasound diagnostic apparatus according to an aspect of the present invention comprises: an ultrasound probe; a position sensor that acquires position information of the ultrasound probe; an image acquisition unit that acquires a plurality of frames of ultrasound images obtained by capturing a blood vessel of a subject by transmitting and receiving ultrasound beams using the ultrasound probe, the plurality of frames of ultrasound images representing a short-axis image of the blood vessel; a three-dimensional image data generation unit that generates three-dimensional ultrasound image data of the subject based on the position information of the ultrasound probe acquired by the position sensor and the plurality of frames of ultrasound images acquired by the image acquisition unit; a centerline acquisition unit that acquires a centerline of the blood vessel in a three-dimensional space based on the three-dimensional ultrasound image data generated by the three-dimensional image data generation unit; a meandering degree calculation unit that calculates a meandering degree of the centerline acquired by the centerline acquisition unit in a transverse diameter direction of the blood vessel, the transverse diameter direction being perpendicular to a plane corresponding to the short-axis image of the blood vessel; and a guide unit that guides the ultrasound probe to a range on the centerline based on the meandering degree calculated by the meandering degree calculation unit. Therefore, it is possible to easily acquire an ultrasound image of a blood vessel region suitable for insertion of an insertion object.

### BRIEF DESCRIPTION OF THE DRAWINGS

FIG. 1 is a block diagram showing a configuration of an ultrasound diagnostic apparatus according to Embodiment 1 of the present invention.

FIG. 2 is a block diagram showing an internal configuration of a transmission/reception circuit in Embodiment 1 of the present invention.

FIG. 3 is a block diagram showing an internal configuration of an image generation unit in Embodiment 1 of the present invention.

FIG. 4 is a diagram schematically showing an ultrasound probe that moves on a body surface of a subject.

FIG. 5 is a diagram schematically showing an example of an ultrasound image representing a short-axis image of a blood vessel.

FIG. 6 is a diagram schematically showing an example of an ultrasound image representing a long-axis image of the blood vessel.

FIG. 7 is a diagram schematically showing an example of a meandering blood vessel in an arm of the subject.

FIG. 8 is a diagram showing a transverse diameter direction of the blood vessel.

FIG. 9 is a diagram showing an example of a centerline of the blood vessel and an average line representing an average position of the centerline in the transverse diameter direction.

FIG. 10 is a diagram showing an example of distances of inflection points of the centerline of the blood vessel from the average line.

FIG. 11 is a diagram showing an example of a distance between the inflection points of the centerline of the blood vessel.

FIG. 12 is a flowchart showing an operation of the ultrasound diagnostic apparatus according to Embodiment 1 of the present invention.

FIG. 13 is a block diagram showing a configuration of an ultrasound diagnostic apparatus according to Embodiment 2 of the present invention.

FIG. 14 is a diagram showing an example of a guide for an ultrasound probe superimposed on an optical image.

FIG. 15 is a diagram showing another example of a guide for the ultrasound probe superimposed on an optical image.

FIG. 16 is a block diagram showing a configuration of an ultrasound diagnostic apparatus according to Embodiment 3 of the present invention.

FIG. 17 is a block diagram showing a configuration of an ultrasound diagnostic apparatus according to Embodiment 4 of the present invention.

FIG. 18 is a block diagram showing a configuration of an ultrasound diagnostic apparatus according to Embodiment 5 of the present invention.

### DESCRIPTION OF THE PREFERRED EMBODIMENTS

Hereinafter, embodiments of the present invention will be described based on the accompanying drawings.

Description of configuration requirements below may be made based on a typical embodiment of the present invention, but the present invention is not limited to such an embodiment.

In the present specification, the numerical range represented by “to” means a range including numerical values denoted before and after “to” as a lower limit value and an upper limit value.

In the present specification, the terms “same” and “identical” include an error range generally allowed in the technical field.

### Embodiment 1

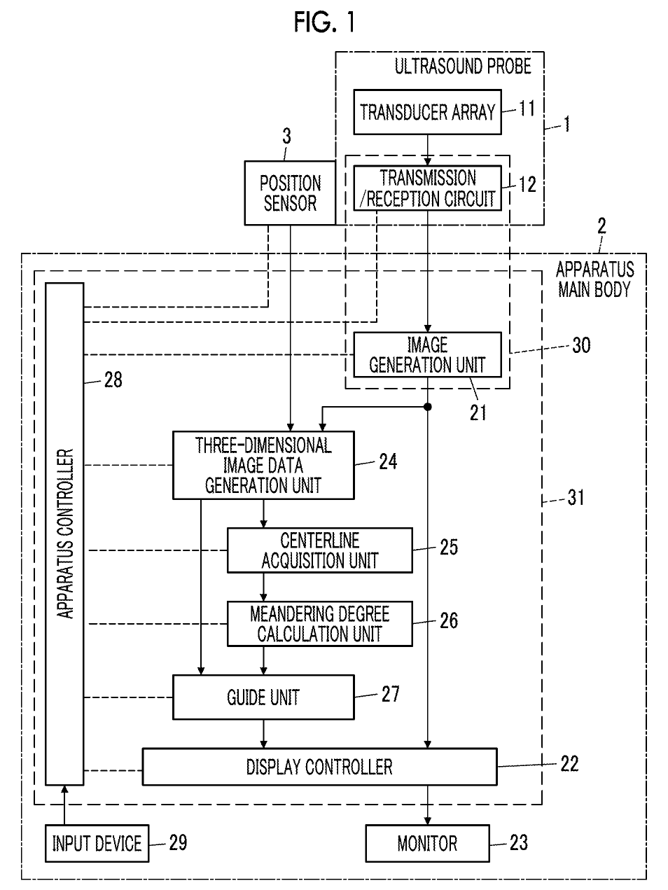

FIG. 1 shows a configuration of an ultrasound diagnostic apparatus according to Embodiment 1 of the present invention. The ultrasound diagnostic apparatus comprises an ultrasound probe 1 and an apparatus main body 2 connected to the ultrasound probe 1. The ultrasound probe 1 and the apparatus main body 2 are connected to each other by so-called wired communication or so-called wireless communication.

The ultrasound probe 1 comprises a transducer array 11 and a transmission/reception circuit 12 connected to the transducer array 11. A position sensor 3 that acquires position information of the ultrasound probe 1 is attached to the ultrasound probe 1.

The apparatus main body 2 comprises an image generation unit 21 connected to the transmission/reception circuit 12 of the ultrasound probe 1. A display controller 22 and a monitor 23 are connected to the image generation unit 21 in this order. In addition, the apparatus main body 2 comprises a three-dimensional image data generation unit 24 connected to the position sensor 3 and the image generation unit 21. A centerline acquisition unit 25 and a meandering degree calculation unit 26 are connected to the three-dimensional image data generation unit 24 in this order. A guide unit 27 is connected to the three-dimensional image data generation unit 24 and the meandering degree calculation unit 26. The guide unit 27 is connected to the display controller 22. In addition, an apparatus controller 28 is connected to the position sensor 3, the transmission/reception circuit 12, the image generation unit 21, the display controller 22, the three-dimensional image data generation unit 24, the centerline acquisition unit 25, the meandering degree calculation unit 26, and the guide unit 27. An input device 29 is connected to the apparatus controller 28.

The transmission/reception circuit 12 and the image generation unit 21 constitute an image acquisition unit 30. In addition, the image generation unit 21, the display controller 22, the three-dimensional image data generation unit 24, the centerline acquisition unit 25, the meandering degree calculation unit 26, the guide unit 27, and the apparatus controller 28 constitute a processor 31 for the apparatus main body 2.

The ultrasound probe 1 is used to capture a so-called ultrasound image that represents a tomographic plane within a subject by transmitting ultrasound beams into the subject while in contact with a body surface of the subject and receiving ultrasound echoes reflected from the inside of the subject.

The transducer array 11 of the ultrasound probe 1 has a plurality of ultrasound transducers arranged one-dimensionally or two-dimensionally. Each of these ultrasound transducers transmits an ultrasound wave in accordance with a drive signal supplied from the transmission/reception circuit 12 and receives an ultrasound echo from the subject to output a signal based on the ultrasound echo. Each ultrasound transducer is configured by forming electrodes at both ends of a piezoelectric body consisting of, for example, a piezoelectric ceramic represented by lead zirconate titanate (PZT), a polymer piezoelectric element represented by poly vinylidene di fluoride (PVDF), a piezoelectric single crystal represented by lead magnesium niobate-lead titanate (PMN-PT), or the like.

The image acquisition unit 30 configured by the transmission/reception circuit 12 and the image generation unit 21 acquires an ultrasound image by transmitting and receiving ultrasound beams by using the ultrasound probe 1.

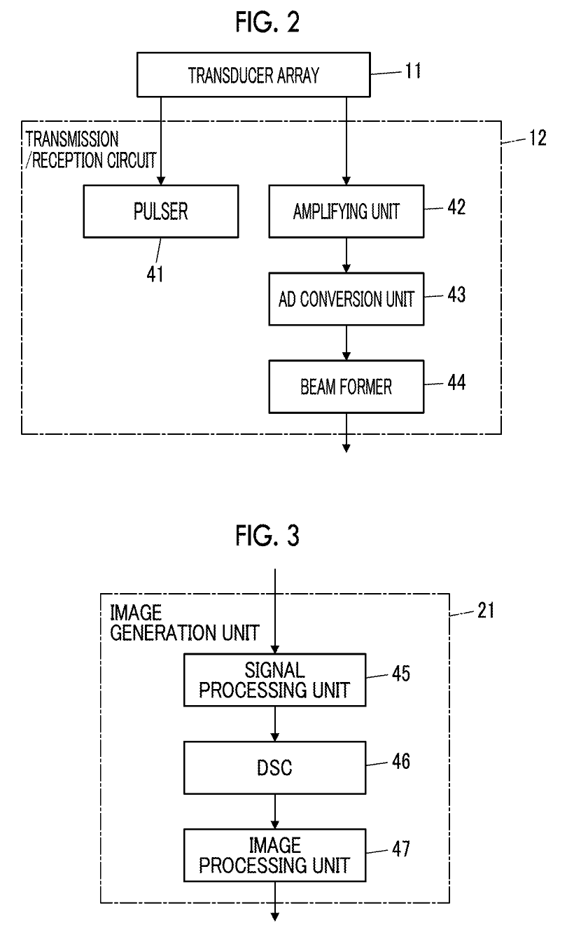

The transmission/reception circuit 12 transmits the ultrasound waves from the transducer array 11 and generates a sound ray signal based on reception signals acquired by the transducer array 11 under control of the apparatus controller 28. As shown in FIG. 2, the transmission/reception circuit 12 includes a pulser 41 connected to the transducer array 11, and an amplification unit 42, an analog-to-digital (AD) conversion unit 43, and a beam former 44 that are sequentially connected in series to the transducer array 11.

The pulser 41 includes, for example, a plurality of pulse generators, adjusts a delay amount of each drive signal based on a transmission delay pattern selected in accordance with a control signal from the apparatus controller 28 so that the ultrasound waves transmitted from the plurality of ultrasound transducers of the transducer array 11 form ultrasound beams, and supplies each drive signal to the plurality of ultrasound transducers. As described above, in a case where a pulsed or continuous wave-like voltage is applied to electrodes of the ultrasound transducer of the transducer array 11, the piezoelectric body expands and contracts to generate pulsed or continuous wave-like ultrasound waves from each of the ultrasound transducers, whereby the ultrasound beam is formed from a combined wave of the ultrasound waves.

The transmitted ultrasound beam is, for example, reflected by a target such as a part of the subject and propagates toward the transducer array 11 of the ultrasound probe 1. The ultrasound echo propagating toward the transducer array 11 in this way is received by each of the ultrasound transducers constituting the transducer array 11. In this case, each of the ultrasound transducers constituting the transducer array 11 expands and contracts by receiving the propagating ultrasound echo to generate a reception signal, which is an electrical signal, and outputs these reception signals to the amplification unit 42.

The amplification unit 42 amplifies the signal input from each of the ultrasound transducers constituting the transducer array 11 and transmits the amplified signal to the AD conversion unit 43. The AD conversion unit 43 converts the signal transmitted from the amplification unit 42 into digital reception data. The beam former 44 performs so-called reception focus processing of applying delays to respective pieces of the reception data received from the AD conversion unit 43 and adding up the results. Through this reception focus processing, a sound ray signal in which each reception data converted by the AD conversion unit 43 is phase-added and the focus of the ultrasound echo is narrowed down is acquired.

As shown in FIG. 3, the image generation unit 21 has a configuration in which a signal processing unit 45, a digital scan converter (DSC) 46, and an image processing unit 47 are sequentially connected in series.

The signal processing unit 45 corrects attenuation by distance of the sound ray signal received from the transmission/reception circuit 12 in accordance with depths of reflection positions of the ultrasound waves using a sound speed value set by the apparatus controller 28 and then performs envelope detection processing on the sound ray signal to generate a B-mode image signal that is tomographic image information related to tissues in the subject.

The DSC 46 converts (raster-converts) the B-mode image signal, which is generated by the signal processing unit 45, into an image signal in accordance with a normal television signal scanning method.

The image processing unit 47 performs various types of necessary image processing such as gradation processing on the B-mode image signal input from the DSC 46, and then transmits the B-mode image signal to the display controller 22 and the three-dimensional image data generation unit 24. Hereinafter, the B-mode image signal that has been subjected to image processing by the image processing unit 47 will be referred to as an ultrasound image.

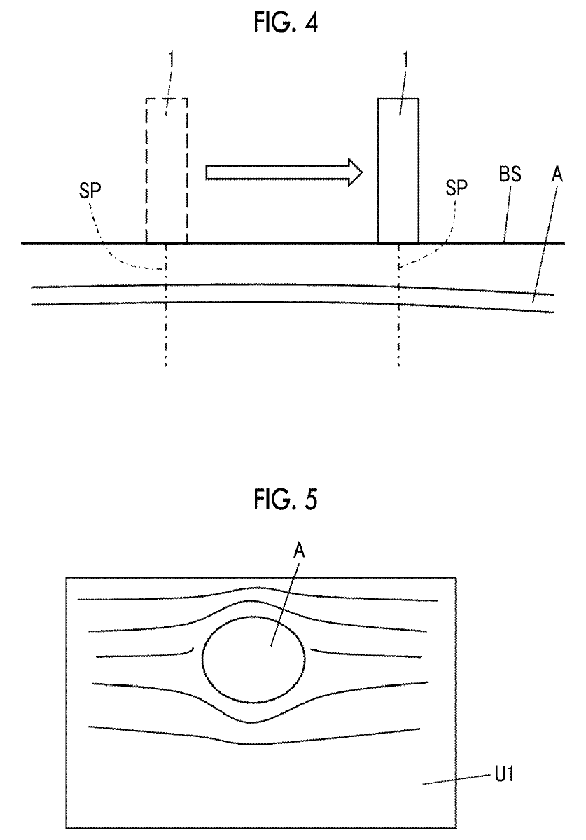

For example, as shown in FIG. 4, a plurality of frames of ultrasound images can be acquired while the ultrasound probe 1 is moved along a blood vessel A in the subject in a state of being in contact with a body surface BS of the subject. In this case, ideally, the orientation of the ultrasound probe 1 is fixed such that a scanning plane SP of the ultrasound probe 1 is perpendicular to a longitudinal direction of the blood vessel A. As a result, for example, as shown in FIG. 5, a plurality of frames of ultrasound images U1 representing a so-called short-axis image that is a cross section of the blood vessel A are acquired.

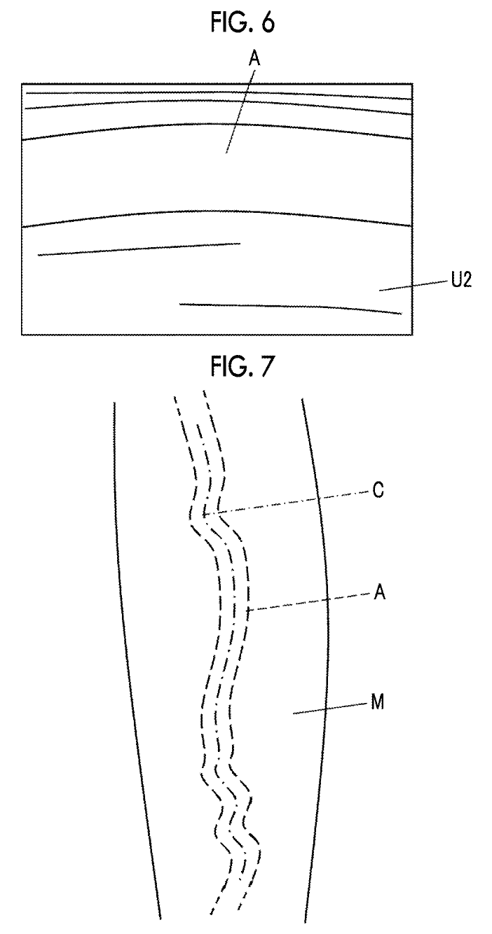

In addition, by adjusting the orientation of the ultrasound probe 1 such that a scanning plane SP parallel to the longitudinal direction of the blood vessel A is obtained, it is also possible to acquire, for example as shown in FIG. 6, an ultrasound image U2 representing a so-called long-axis image that is a longitudinal section of the blood vessel A.

The position sensor 3 is a device that is attached to the ultrasound probe 1 and that acquires the position information of the ultrasound probe 1. The position information of the ultrasound probe 1 acquired by the position sensor 3 can include not only position coordinates of the ultrasound probe 1 in a three-dimensional space but also angle coordinates of the ultrasound probe 1 in the three-dimensional space. As the position sensor 3, for example, a known sensor device such as an acceleration sensor, a gyro sensor, a magnetic sensor, and a global positioning system (GPS) sensor can be used.

The three-dimensional image data generation unit 24 generates three-dimensional ultrasound image data of the subject based on the plurality of frames of ultrasound images U1 acquired by the image acquisition unit 30 and representing the short-axis image of the blood vessel A while the ultrasound probe 1 moves on the body surface BS of the subject with the orientation of the ultrasound probe 1 fixed, and the position information of the ultrasound probe 1 continuously acquired by the position sensor 3 while the plurality of frames of ultrasound images U1 are captured. The three-dimensional ultrasound image data includes a three-dimensional structure of the blood vessel A.

In a case of generating the three-dimensional ultrasound image data, the three-dimensional image data generation unit 24 specifies the three-dimensional structure of the blood vessel A in the three-dimensional ultrasound image data by extracting the short-axis image of the blood vessel A from the plurality of frames of ultrasound images U1 using an algorithm such as so-called binarization processing or so-called template matching, and using the extraction result.

The centerline acquisition unit 25 acquires a centerline C of the blood vessel A, for example, as schematically shown in FIG. 7 based on the three-dimensional ultrasound image data generated by the three-dimensional image data generation unit 24. FIG. 7 shows a blood vessel A in an arm M of the subject. The centerline acquisition unit 25 can acquire the centerline C of the blood vessel A in the three-dimensional space by, for example, performing so-called thinning processing on the three-dimensional structure of the blood vessel A specified by the three-dimensional image data generation unit 24.

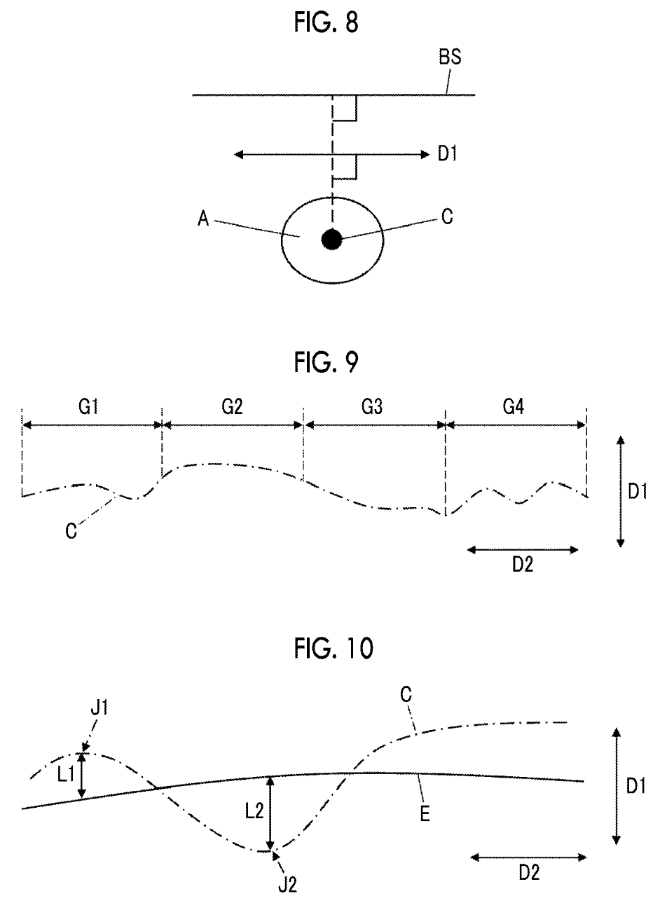

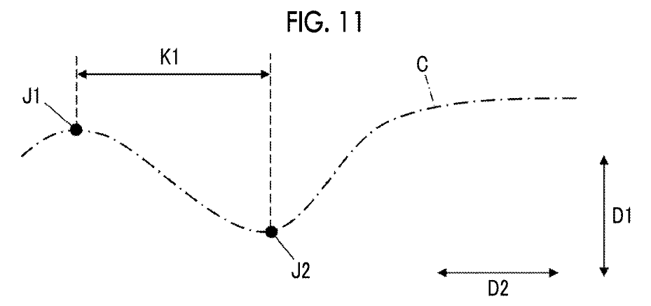

The meandering degree calculation unit 26 calculates a transverse diameter direction of the blood vessel A perpendicular to a plane corresponding to the short-axis image of the blood vessel A, and calculates a meandering degree along the transverse diameter direction of the centerline C. By referring to the three-dimensional ultrasound image data, the meandering degree calculation unit 26 can define a direction along the body surface BS as a transverse diameter direction D1 of the blood vessel A in a plane that corresponds to the short-axis image of the blood vessel A and that is perpendicular to the body surface BS as shown schematically in FIG. 8 by regarding the body surface BS as a plane, for example.

By defining a direction orthogonal to the transverse diameter direction D1 of the blood vessel A and a depth direction of the blood vessel A as a traveling direction D2 of the blood vessel A, the meandering degree calculation unit 26 can divide the centerline C into a plurality of sections G1, G2, G3, and G4 having a predetermined length along the traveling direction D2 of the blood vessel A, and calculate a meandering degree for each of the sections G1 to G4, for example, as shown in FIG. 9. The number of the divided sections is not limited to four, and may be two, three, or five or more depending on a length of the centerline C. A length of each of the sections G1 to G4 can be set to, for example, about a width orthogonal to the depth direction of the ultrasound image U2 representing the long-axis image of the blood vessel A, for example.

The meandering degree calculation unit 26 can calculate, with respect to the centerline C, an average line E representing an average position of the centerline C in the transverse diameter direction D1 of the blood vessel A, for each of the plurality of sections G1 to G4, for example, as shown in FIG. 10 by applying a so-called least squares method to each of the plurality of sections G1 to G4. In this case, the meandering degree calculation unit 26 can calculate a plurality of inflection points J1 and J2 of the centerline C, and calculate distances L1 and L2 between the plurality of inflection points J1 and J2 and the average line E in the transverse diameter direction D1. The meandering degree calculation unit 26 can calculate, for example, the number of the inflection points J1 and J2 at which the distances L1 and L2 are equal to or greater than a predetermined distance threshold value for each of the plurality of sections G1 to G4, as the meandering degree. It can be determined that the larger the number of the inflection points J1 and J2 at which the distances L1 and L2 are equal to or greater than the distance threshold value, the greater the number of portions at which the blood vessel A meanders, and conversely, it can be determined that the fewer the number of the inflection points J1 and J2 at which the distances L1 and L2 are equal to or greater than the distance threshold value, the fewer the number of portions at which the blood vessel A meanders.

In addition, the meandering degree calculation unit 26 can calculate, for example, a maximum value of a reciprocal of a distance K1 between the inflection points J1 and J2 of the centerline C in the traveling direction D2 for each of the plurality of sections G1 to G4, as the meandering degree. The larger the value of the reciprocal of the interval K1, the narrower the meandering interval along the traveling direction D2, and thus it can be determined that there are many portions at which the blood vessel A meanders, and conversely, the fewer the value of the reciprocal of the interval K1, the wider the meandering interval along the traveling direction D2, and thus it can be determined that there are fewer portions at which the blood vessel A meanders.

The guide unit 27 guides the ultrasound probe 1 to a range on the centerline C suitable for insertion of an insertion object, that is, a blood vessel region suitable for the insertion of the insertion object, based on the meandering degree calculated by the meandering degree calculation unit 26. The guide unit 27 can guide the ultrasound probe 1 to, for example, a range on the centerline C where the meandering degree calculated by the meandering degree calculation unit 26 is equal to or less than a predetermined meandering degree threshold value, for example, a section where the meandering degree is equal to or less than the meandering degree threshold value among the plurality of sections G1 to G4.

The guide unit 27 can guide the ultrasound probe 1 by, for example, displaying, on the monitor 23, a so-called schema, which is a human body model diagram, and a so-called probe mark disposed at a location on the human body model diagram corresponding to the blood vessel region suitable for the insertion of the insertion object in a superimposed manner, by displaying, on the monitor 23, a difference value between the current position and inclination angle of the ultrasound probe 1 and the position of the blood vessel region suitable for the insertion of the insertion object and a recommended inclination angle of the ultrasound probe 1 at the position, or by displaying a direction from the current position of the ultrasound probe 1 to the blood vessel region suitable for the insertion of the insertion object on the monitor 23.

In a case where the meandering degree is equal to or less than the meandering degree threshold value in two or more of the plurality of sections G1 to G4 of the centerline C, the guide unit 27 can guide the ultrasound probe 1 to a section with the smallest meandering degree, for example.

Here, in medical settings, an insertion object such as a so-called biopsy needle or a so-called catheter may be inserted into the blood vessel A of the subject to perform an examination or treatment on the subject. In this case, in order to non-invasively check a positional relationship between the blood vessel A in the subject and the insertion object, a procedure of capturing an ultrasound image U1 or U2 showing the blood vessel A in the subject and the insertion object by using an ultrasound diagnostic apparatus is known. In such a procedure, the short-axis image and the long-axis image of the blood vessel A are alternately captured to observe the state in which the insertion object is inserted into the blood vessel A, but, in a case where the blood vessel A meanders in the transverse diameter direction D1, the blood vessel A is depicted in a state of being interrupted in a case where the long-axis image is captured, resulting in problems such as inability to accurately understand the state of the insertion object inserted into the blood vessel A. Therefore, it is preferable that the portion of the blood vessel A into which the insertion object is inserted does not meander in the transverse diameter direction D1.

The guide unit 27 guides the ultrasound probe 1 to a range where the blood vessel A does not meander or has minimal meandering in the transverse diameter direction D1, so that the user of the ultrasound diagnostic apparatus, such as a doctor, can proceed with the procedure while accurately understanding the positional relationship between the insertion object inserted into the subject and the blood vessel A by inserting the insertion object into the guided position.

In addition, the guide unit 27 can acquire a depth of the blood vessel A from the body surface BS of the subject over the entire centerline C, for example, by referring to the three-dimensional ultrasound image data generated by the three-dimensional image data generation unit 24, and guide the ultrasound probe 1 to a range where the meandering degree is equal to or less than a predetermined meandering degree threshold value and the depth of the blood vessel A is equal to or less than a predetermined depth threshold value. Here, the guide unit 27 can measure the shortest distance along the depth direction between the body surface BS of the subject and the blood vessel A at each point in the traveling direction D2 of the blood vessel A as the depth of the blood vessel A. In a case where the blood vessel A is located at a deep position, it is difficult to insert the insertion object into the blood vessel A. Therefore, in a case of inserting the insertion object into the blood vessel A, usually, a blood vessel A that is present within 2.0 cm, preferably within 1.5 cm from the body surface BS is often selected as a target for the insertion. Therefore, the depth threshold value can be set to, for example, 2.0 cm, preferably 1.5 cm.

In addition, in a case where the insertion object is a catheter, in a case where an inner diameter of the blood vessel A is smaller than an outer diameter of the catheter, it is impossible to insert the catheter into the blood vessel A, and, in a case where the inner diameter of the blood vessel A is too large compared to the outer diameter of the catheter, the catheter may not be able to sufficiently expand the blood vessel A, resulting in a reduced treatment effect. Therefore, in medical settings or the like, the inner diameter of blood vessel A that is recommended for insertion relative to the outer diameter of the catheter may be predetermined. Therefore, the guide unit 27 can acquires an inner diameter of the blood vessel A over the entire centerline C, for example, by referring to the three-dimensional ultrasound image data generated by the three-dimensional image data generation unit 24, and guide the ultrasound probe 1 to a range where the meandering degree is equal to or less than a meandering degree threshold value and the inner diameter of the blood vessel A is closest to a predetermined recommended inner diameter value. The recommended inner diameter value can be set to, for example, about three times the outer diameter of the catheter inserted into the subject.

Under the control of the apparatus controller 28, the display controller 22 performs predetermined processing on the ultrasound images U1 and U2 acquired by the image acquisition unit 30, the information on the guide from the guide unit 27, and the like, and displays them on the monitor 23.

The monitor 23 displays the ultrasound images U1 and U2 and the like under the control of the display controller 22, and includes, for example, a display device such as a liquid crystal display (LCD) or an organic electroluminescence display (organic EL display).

The apparatus controller 28 controls each unit of the apparatus main body 2, the transmission/reception circuit 12 of the ultrasound probe 1, and the position sensor 3 based on a control program and the like stored in advance.

The input device 29 is a device for the user to perform an input operation, and includes, for example, a device such as a keyboard, a mouse, a track ball, a touch pad, and a touch sensor disposed on the monitor 23 in a superimposed manner.

In the present embodiment, each process is executed by any computer. In addition, any computer may execute these processes using the processor 31 as hardware, a program as software, or a combination thereof. In that case, the processor 31 is configured to execute various processes in the present embodiment in cooperation with the program, and can function as each unit or each means in the present embodiment. In addition, the order in which the processes are executed by the processor 31 is not limited to the order described above and may be changed as appropriate. Any computer may be a general-purpose computer, a computer for a specific use, a workstation, or another system capable of executing each process.

The processor 31 may be configured by one or more pieces of hardware, and the type of hardware is not limited. For example, the processor 31 may be configured by a programmable logic device such as a central processing unit (CPU), a micro processing unit (MPU), or a field programmable gate array (FPGA), a dedicated circuit for executing specific processing such as an application specific integrated circuit (ASIC), or hardware such as a graphics processing unit (GPU) or a neural processing unit (NPU). In addition, the types of hardware may be a combination of different types of hardware. In a case where a plurality of pieces of hardware are configured to execute one or a plurality of processes of the processor 31, the plurality of pieces of hardware may be present in devices physically separated from each other, or may be present in the same device. In addition, in any of the embodiments, the order of each processing executed by the processor 31 is not limited to the above order and may be changed as appropriate. The hardware is configured by an electric circuit (circuitry) in which circuit elements such as semiconductor elements are combined.

Further, the program may be software such as firmware or a microcode. In addition, the program may be, for example, a program module group, and each function thereof may be realized by the processor 31 configured to execute each function. The program may be a program code or a plurality of code segments stored in one or a plurality of non-transitory computer-readable media (for example, a storage medium or other storage). The program may be divided and stored in a plurality of non-transitory computer-readable media present in devices physically separated from each other. The program code or the code segment may represent any combination of a procedure, a function, a subprogram, a routine, a subroutine, a module, a software package, a class, an instruction, a data structure, or a program statement. The program code or the code segment may be connected to another code segment or a hardware circuit by transmitting and receiving information, data, an argument, a parameter, or memory contents.

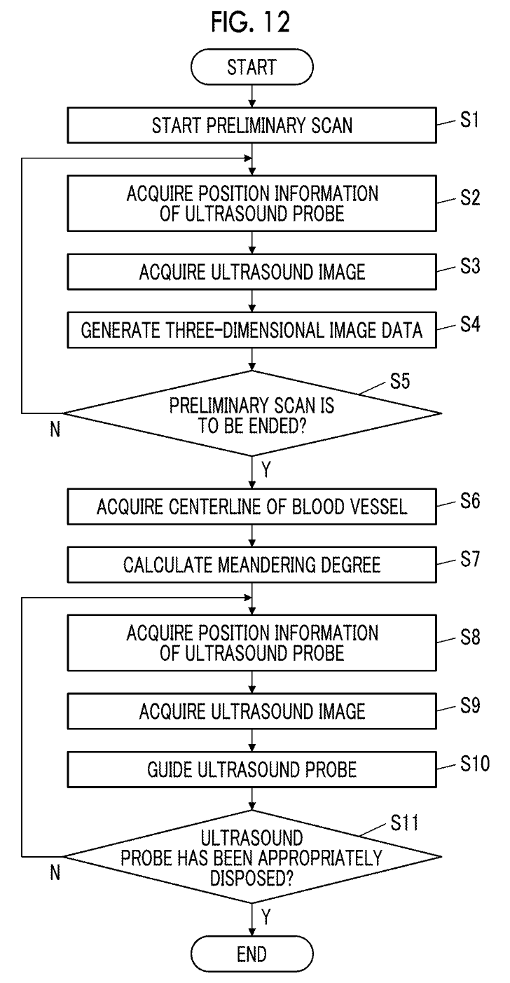

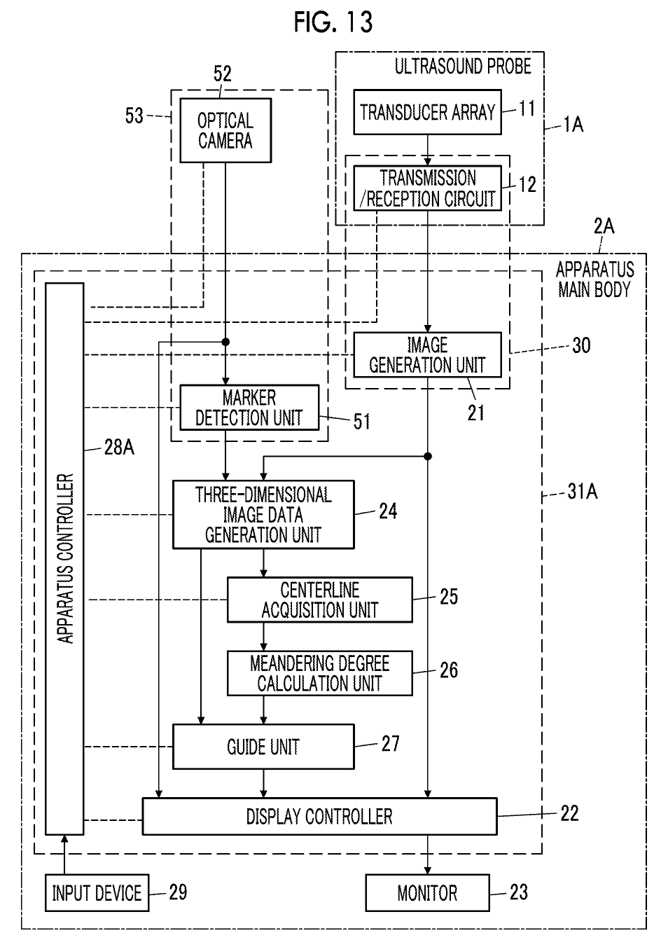

Hereinafter, an operation of the ultrasound diagnostic apparatus according to Embodiment 1 will be described with reference to a flowchart shown in FIG. 12.

In step S1, for example, a mode of performing a preliminary scan is started based on an instruction from the user via the input device 29. The user moves the ultrasound probe 1 along the blood vessel A in a state where the ultrasound probe 1 is in contact with the body surface BS of the subject.

In step S2, the position sensor 3 acquires the current position information of the ultrasound probe 1. The position information of the ultrasound probe 1 acquired in step S2 is transmitted to the three-dimensional image data generation unit 24.

In step S3, the image acquisition unit 30 acquires, for example, the ultrasound image U1 representing the short-axis image of the blood vessel A in the subject as shown in FIG. 5. In this case, under the control of the apparatus controller 28, the transmission and reception of the ultrasound waves from the plurality of transducers of the transducer array 11 are started in accordance with the drive signal from the pulser 41 of the transmission/reception circuit 12 of the ultrasound probe 1, the ultrasound echo from the inside of the subject is received by the plurality of transducers of the transducer array 11, and the reception signal as the analog signal is output to the amplification unit 42, is amplified, and then is subjected to the AD conversion via the AD conversion unit 43 to acquire the reception data.

The reception focus processing is performed on the reception data by the beam former 44, the sound ray signal generated by the reception focusing processing is transmitted to the image generation unit 21 of the apparatus main body 2, and thus the ultrasound image U1 is generated by the image generation unit 21. In this case, the signal processing unit 45 of the image generation unit 21 performs the correction of the attenuation in accordance with the depth of the reflection position of the ultrasound waves and the envelope detection processing on the sound ray signal, the DSC 46 performs the conversion into the image signal in accordance with the normal television signal scanning method, and the image processing unit 47 performs various types of necessary image processing, such as gradation processing. The ultrasound image U1 representing the short-axis image of the blood vessel A generated in step S3 in this way is transmitted to the display controller 22 and displayed on the monitor 23, and is also transmitted to the three-dimensional image data generation unit 24.

In step S4, the three-dimensional image data generation unit 24 generates three-dimensional ultrasound image data of the inside of the subject based on the position information of the ultrasound probe 1 acquired in step S2 and the ultrasound image U1 representing the short-axis image of the blood vessel A acquired in step S3. In this case, the three-dimensional image data generation unit 24 extracts the short-axis image of the blood vessel A captured in the ultrasound image U1 acquired in step S3, and specifies the three-dimensional structure of the blood vessel A in the three-dimensional ultrasound image data.

In step S5, the apparatus controller 28 determines whether or not to end the preliminary scan. The apparatus controller 28 can determine to end the preliminary scan, for example, in a case where the user determines that the ultrasound image U1 representing the short-axis image of the blood vessel A is sufficiently acquired and inputs an instruction to end the preliminary scan via the input device 29. The apparatus controller 28 can determine to continue the preliminary scan, for example, in a case where the user determines that the ultrasound image U1 is not sufficiently acquired and does not input any instruction via the input device 29.

In a case where it is determined to continue the preliminary scan in step S5, the process returns to step S2. As described above, the processes of steps S2 to S5 are repeated as long as it is determined in step S5 to continue the preliminary scan. As a result, while the ultrasound probe 1 moves on the body surface BS of the subject along the blood vessel A, the position information of the ultrasound probe 1 is continuously acquired by the position sensor 3, and a plurality of consecutive frames of ultrasound images U1 are acquired. Each time the position information of the ultrasound probe 1 is acquired and the ultrasound image U1 is acquired, data related to the three-dimensional structure inside the subject corresponding to the newly acquired ultrasound image U1 is cumulatively added to the three-dimensional ultrasound image data in step S4, and three-dimensional ultrasound image data representing the three-dimensional structure inside the subject is constructed.

In a case where it is determined in step S5 to end the preliminary scan, the process proceeds to step S6. In step S6, the centerline acquisition unit 25 acquires, for example, the centerline C of the blood vessel A, for example, as schematically shown in FIG. 7 based on the three-dimensional ultrasound image data acquired in step S4. The centerline acquisition unit 25 can acquire the centerline C of the blood vessel A in the three-dimensional space by, for example, performing thinning processing on the three-dimensional structure of the blood vessel A specified by the three-dimensional image data generation unit 24 in step S4.

In step S7, the meandering degree calculation unit 26 calculates the meandering degree of the centerline C acquired in step S6 in the transverse diameter direction D1 of the blood vessel A. The meandering degree calculation unit 26 can define a direction parallel to the body surface BS as the transverse diameter direction D1 in any plane parallel to the short-axis image of the blood vessel A in the three-dimensional ultrasound image data and orthogonal to the body surface BS, for example, as shown in FIG. 8, by regarding the body surface BS of the subject as a plane.

Further, for example, as shown in FIG. 9, the meandering degree calculation unit 26 can divide the centerline C into a plurality of sections G1 to G4 having a predetermined length along the traveling direction D2 of the blood vessel A, and calculate the meandering degree in each section. The length of each of the sections G1 to G4 can be set to, for example, about a width orthogonal to the depth direction of the ultrasound image U2 representing the long-axis image of the blood vessel A, for example.

The meandering degree calculation unit 26 can calculate, with respect to the centerline C, an average line E representing an average position of the centerline C in the transverse diameter direction D1, for each point of the centerline C in the traveling direction D2, as shown in FIG. 10, by applying a least squares method to each of the plurality of sections G1 to G4. The meandering degree calculation unit 26 can further calculate a plurality of inflection points J1 and J2 of the centerline C, and calculate distances L1 and L2 between the plurality of inflection points J1 and J2 and the average line E. The meandering degree calculation unit 26 can calculate, for example, the number of the inflection points J1 and J2 at which the distances L1 and L2 are equal to or greater than a predetermined distance threshold value for each of the plurality of sections G1 to G4, as the meandering degree.

In addition, the meandering degree calculation unit 26 can calculate, for example, a maximum value of a reciprocal of a distance K1 between the inflection points J1 and J2 along the traveling direction D2 of the blood vessel A for each of the plurality of sections G1 to G4, as the meandering degree.

In step S8, the position sensor 3 acquires the current position information of the ultrasound probe 1 in the same manner as in step S2.

In step S9, the image acquisition unit 30 acquires the ultrasound image U1 representing the short-axis image of the blood vessel A or the ultrasound image U2 representing the long-axis image of the blood vessel A in the same manner as in step S3. The ultrasound image U1 or U2 acquired in step S9 is displayed on the monitor 23.

In step S10, the guide unit 27 guides the ultrasound probe 1 to a range on the centerline C based on the meandering degree of the centerline C calculated in step S7. In this case, the guide unit 27 can guide the ultrasound probe 1 to, for example, a section where the meandering degree is equal to or less than the meandering degree threshold value among the plurality of sections G1 to G4 on the centerline C. The guide unit 27 can display, for example, the guide for the ultrasound probe 1 on the monitor 23. By checking the guide provided by the guide unit 27, the user can move the ultrasound probe 1 such that the ultrasound probe 1 is disposed in a section where the meandering degree is equal to or less than the meandering degree threshold value.

In step S11, the apparatus controller 28 determines whether or not the ultrasound probe 1 has been appropriately disposed in the blood vessel region suitable for the insertion of the insertion object. By referring to, for example, the position information of the ultrasound probe 1 acquired in step S8, the apparatus controller 28 can determine that the ultrasound probe 1 has been appropriately disposed in a case where the ultrasound probe 1 is disposed at the position of the blood vessel region guided in step S10. In addition, the apparatus controller 28 can determine that the ultrasound probe 1 has not been appropriately disposed in a case where the ultrasound probe 1 is not disposed at the position of the blood vessel region guided in step S10.

In a case where it is determined in step S11 that the ultrasound probe 1 has not been appropriately disposed, the process returns to step S8. As described above, the processes of steps S8 to S11 are repeated as long as it is determined in step S11 that the ultrasound probe 1 has not been appropriately disposed. During this time, the user moves the ultrasound probe 1 toward the position of the blood vessel region guided in step S10 while checking the guide in step S10.

In a case where it is determined in step S11 that the ultrasound probe 1 has been appropriately disposed, the operation of the ultrasound diagnostic apparatus according to the flowchart of FIG. 12 is completed. In this way, the user can easily acquire the ultrasound images U1 and U2 of the blood vessel region suitable for the insertion of the insertion object. The user can accurately and safely insert the insertion object into the blood vessel A while checking the ultrasound images U1 and U2.

As described above, with the ultrasound diagnostic apparatus of Embodiment 1 of the present invention, the three-dimensional image data generation unit 24 generates the three-dimensional ultrasound image data based on the position information of the ultrasound probe 1 acquired by the position sensor 3 and the plurality of frames of ultrasound images U1 acquired by the image acquisition unit 30 and representing the short-axis image of the blood vessel A, the centerline acquisition unit 25 acquires the centerline C of the blood vessel A in the three-dimensional space based on the three-dimensional ultrasound image data, the meandering degree calculation unit 26 calculates the meandering degree along the transverse diameter direction D1 of the centerline C, and the guide unit 27 guides the ultrasound probe 1 to the range on the centerline C based on the meandering degree. Therefore, it is possible to easily acquire the ultrasound images U1 and U2 of the blood vessel region suitable for the insertion of the insertion object.

In addition, a case has been described in which the transmission/reception circuit 12 is provided in the ultrasound probe 1, but the transmission/reception circuit 12 may be provided in the apparatus main body 2.

In addition, a case has been described in which the image generation unit 21 is provided in the apparatus main body 2, but the image generation unit 21 may be provided in the ultrasound probe 1.

The apparatus main body 2 may be a so-called stationary type, a portable type that is easily carried, or a so-called handheld type that is configured by, for example, a smartphone or a tablet type computer. In this way, the type of the device constituting the apparatus main body 2 is not particularly limited.

In addition, the position information of the ultrasound probe 1 acquired by the position sensor 3 can be configured by only the position coordinates of the ultrasound probe 1 in the three-dimensional space. However, since the position information includes not only the position coordinates of the ultrasound probe 1 in the three-dimensional space but also angle coordinates of the ultrasound probe 1 in the three-dimensional space, the three-dimensional image data generation unit 24 can generate the three-dimensional ultrasound image data with higher accuracy than in a case where the position information is configured by only the position coordinates.

In addition, in a case of acquiring the plurality of frames of ultrasound images U1 used to generate the three-dimensional ultrasound image data, it is ideal for the ultrasound probe 1 to be placed perpendicular to the body surface BS of the subject in order to generate accurate three-dimensional ultrasound image data. Therefore, for example, the apparatus controller 28 can determine whether or not the ultrasound probe 1 is placed approximately perpendicular to the body surface BS of the subject by referring to the angle coordinates in the three-dimensional space of the ultrasound probe 1 included in the position information of the ultrasound probe 1, and can provide a warning to the user via the monitor 23 in a case where the ultrasound probe 1 is not placed approximately perpendicular to the body surface BS of the subject. Here, the term “approximately perpendicular” means that the ultrasound probe 1 is in a certain angle range centered on 90 degrees, for example, in a range of 85 degrees to 95 degrees with respect to the body surface BS.

A case has been described in which the guide unit 27 displays the content of the guide for the ultrasound probe 1 on the monitor 23, but the method of the guide is not particularly limited to this. For example, in a case where the ultrasound diagnostic apparatus comprises a speaker (not shown), the guide unit 27 can guide the ultrasound probe 1 by a voice via the speaker.

### Embodiment 2

A case has been described in which the position sensor 3 is attached to the ultrasound probe 1, but the position sensor 3 may be independent of the ultrasound probe 1 as long as it can acquire the position information of the ultrasound probe 1.

An ultrasound diagnostic apparatus of Embodiment 2 differs from the ultrasound diagnostic apparatus shown in FIG. 1 in that an ultrasound probe 1A, on which a marker usable as a so-called Augmented Reality (AR) marker such as a so-called Augmented Reality University of Cordoba (ArUco) is provided, is provided instead of the ultrasound probe 1, an apparatus main body 2A, in which a marker detection unit 51 is added, is provided instead of the apparatus main body 2, and a position sensor 53 configured by an optical camera 52 and the marker detection unit 51 of the apparatus main body 2A, the position sensor 53 being disposed apart from the ultrasound probe 1A, is provided instead of the position sensor 3 attached to the ultrasound probe 1.

The apparatus main body 2A in Embodiment 2 differs from the apparatus main body 2 in Embodiment 1 in that the marker detection unit 51 is added and an apparatus controller 28A is provided instead of the apparatus controller 28. The marker detection unit 51 is connected to the optical camera 52. In addition, the marker detection unit 51 is connected to the three-dimensional image data generation unit 24 and the apparatus controller 28A. The image generation unit 21, the display controller 22, the three-dimensional image data generation unit 24, the centerline acquisition unit 25, the meandering degree calculation unit 26, the guide unit 27, the apparatus controller 28A, and the marker detection unit 51 constitute a processor 31A for the apparatus main body 2A.

The optical camera 52 is connected to the apparatus main body 2A by wired communication or wireless communication, and acquires an optical image in which the ultrasound probe 1A is captured, under the control of the apparatus controller 28A. The optical camera 52 includes, for example, an image sensor such as a so-called charge coupled device (CCD) image sensor or a so-called a complementary metal-oxide-semiconductor (CMOS) image sensor. The optical camera 52 can be fixedly disposed at a position where the marker of the ultrasound probe 1A can be clearly imaged, for example. In addition, the optical camera 52 can also be fixedly disposed on a part of a body of a user, for example, a head of the user. The optical image acquired by the optical camera 52 is transmitted to the marker detection unit 51.

The marker detection unit 51 acquires position information of the ultrasound probe 1A by detecting the marker captured in the optical image acquired by the optical camera 52. The marker detection unit 51 can detect the marker and acquire the position information of the ultrasound probe 1A by using a known algorithm for reading a figure used as the AR marker. For example, in a case where the marker represents ArUco, the marker can be detected and the position information of the ultrasound probe 1A can be acquired by using an algorithm for ArUco included in OpenCV (registered trademark) that is a library.

The three-dimensional image data generation unit 24 generates three-dimensional ultrasound image data of the inside of the subject based on the plurality of frames of ultrasound images U1 acquired by the image acquisition unit 30 and representing the short-axis image of the blood vessel A and the position information of the ultrasound probe 1A acquired by the position sensor 53.

The centerline acquisition unit 25 acquires the centerline C of the blood vessel A based on the three-dimensional ultrasound image data, the meandering degree calculation unit 26 calculates the meandering degree of the centerline C, and the guide unit 27 guides the ultrasound probe 1A based on the meandering degree.

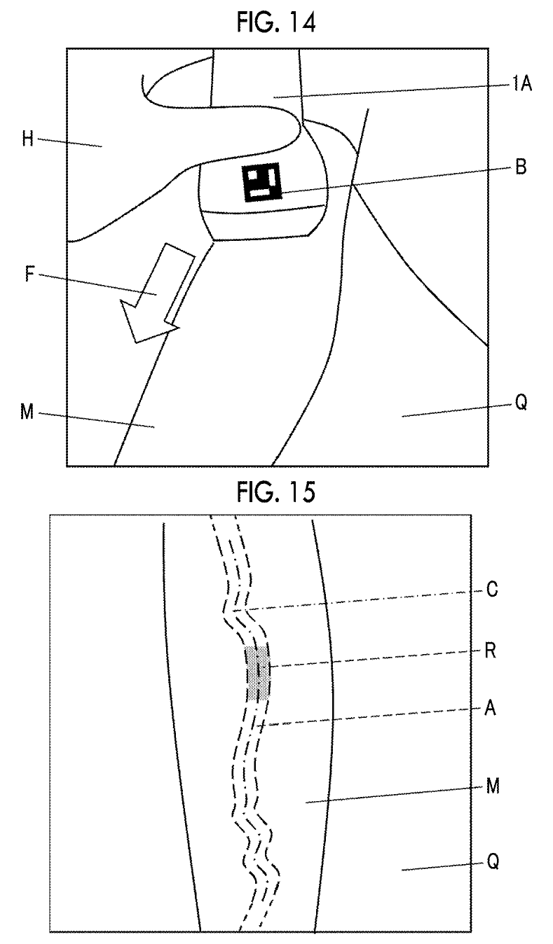

The guide unit 27 can also display the guide for the ultrasound probe 1A on the monitor 23 by superimposing the guide for the ultrasound probe 1A on the optical image acquired by the optical camera 52 based on the position information of the ultrasound probe 1A acquired by the marker detection unit 51. In this case, the guide unit 27 can superimpose, on an optical image Q, an arrow F representing a direction in which the ultrasound probe 1A has to be moved toward the blood vessel region suitable for the insertion of the insertion object, for example, as shown in FIG. 14. FIG. 14 shows a state in which the ultrasound probe 1A on which a marker B is provided and which is gripped by a hand H of the user is disposed on the arm M of the subject. In addition, the guide unit 27 can also highlight a blood vessel region R suitable for the insertion of the insertion object in the optical image Q as the guide for the ultrasound probe 1A, for example, as shown in FIG. 15.

The user can easily position the ultrasound probe 1A in the blood vessel region R suitable for the insertion of the insertion object by checking the guide for the ultrasound probe 1A superimposed on the optical image Q.

As described above, even in a case where the position sensor 53 is configured by the optical camera 52 and the marker detection unit 51 as in the ultrasound diagnostic apparatus of Embodiment 2, the guide unit 27 guides the ultrasound probe 1A to the range on the centerline C based on the meandering degree as in a case where the position sensor 3 is attached to the ultrasound probe 1 as in Embodiment 1, so that the ultrasound images U1 and U2 of the blood vessel region R suitable for the insertion of the insertion object can be easily acquired.

As an example of the position sensor independent of the ultrasound probe 1A, the position sensor 53 configured by the marker detection unit 51 and the optical camera 52 has been described, but the type of the position sensor independent of the ultrasound probe 1A is not particularly limited to this, for example. For example, although not shown, the position sensor can also be configured by a so-called distance-measuring sensor that is independent of the ultrasound probe 1A and an analysis unit that analyzes a signal acquired by the distance-measuring sensor. The analysis unit can acquire the position information of the ultrasound probe 1A by using, for example, a method disclosed in “ZHAO, Mingmin, et al., Through-wall human pose estimation using radio signals, In: Proceedings of the IEEE Conference on Computer Vision and Pattern Recognition, 2018, pp. 7356 to 7365”, “VASILEIADIS, Manolis; BOUGANIS, Christos-Savvas; TZOVARAS, Dimitrios, Multi-person 3D pose estimation from 3D cloud data using 3D convolutional neural networks, Computer Vision and Image Understanding, 2019, 185: 12 to 23”, “JIANG, Wenjun, et al., Towards 3D human pose construction using WiFi, In: Proceedings of the 26th Annual International Conference on Mobile Computing and Networking, 2020, pp. 1 to 14”, or “WANG, Fei, et al., Person-in-WiFi: Fine-grained person perception using WiFi, In: Proceedings of the IEEE/CVF International Conference on Computer Vision, 2019, pp. 5452 to 5461”.

### Embodiment 3

In order to acquire highly accurate three-dimensional ultrasound image data and to accurately guide the ultrasound probe 1 to the blood vessel region R suitable for the insertion of the insertion object, ideally, it is desirable that the posture of the subject does not change between the start of acquisition of the plurality of frames of ultrasound images U1 for generating the three-dimensional ultrasound image data and the insertion of the insertion object into the subject. However, the posture of the subject may change due to some reason. Therefore, in order to respond to the change in the posture of the subject, the ultrasound diagnostic apparatus can use the relative position with respect to the part of the subject as the position information of the ultrasound probe 1.

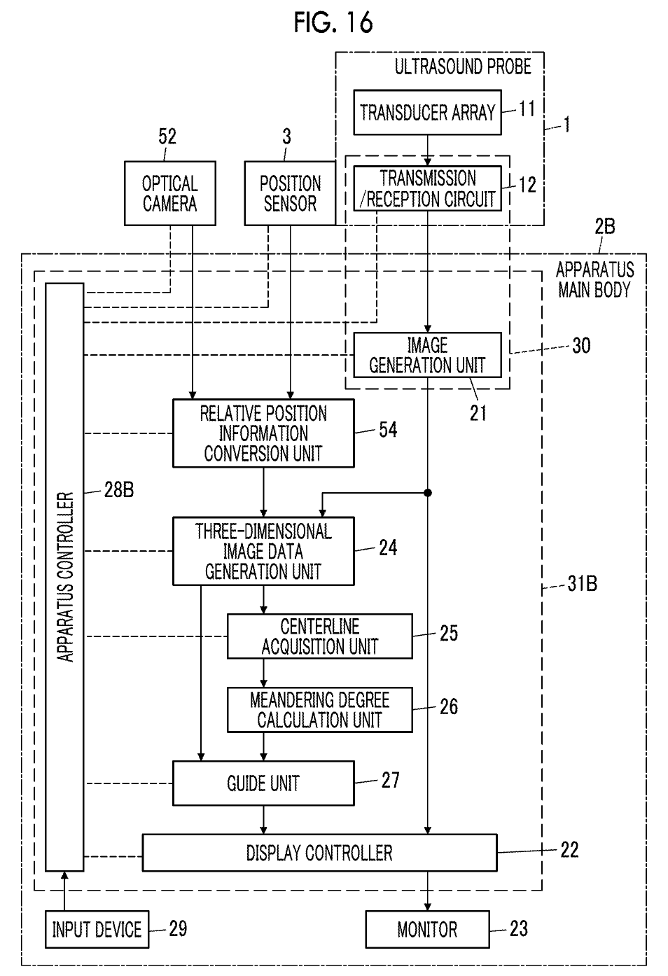

FIG. 16 shows a configuration of an ultrasound diagnostic apparatus of Embodiment 3. The ultrasound diagnostic apparatus of Embodiment 3 differs from the ultrasound diagnostic apparatus according to Embodiment 1 shown in FIG. 1 in that an apparatus main body 2B is provided instead of the apparatus main body 2 and the optical camera 52 is further added. The optical camera 52 is the same as the optical camera 52 in Embodiment 2. The apparatus main body 2B in Embodiment 3 differs from the apparatus main body 2 in Embodiment 1 in that a relative position information conversion unit 54 is further provided and an apparatus controller 28B is provided instead of the apparatus controller 28.

The relative position information conversion unit 54 is connected to the position sensor 3 and the optical camera 52. The relative position information conversion unit 54 is connected to the three-dimensional image data generation unit 24 and the apparatus controller 28B. In addition, the image generation unit 21, the display controller 22, the three-dimensional image data generation unit 24, the centerline acquisition unit 25, the meandering degree calculation unit 26, the guide unit 27, the apparatus controller 28B, and relative position information conversion unit 54 constitute a processor 31B for the apparatus main body 2B.

The relative position information conversion unit 54 converts the position information of the ultrasound probe 1 acquired by the position sensor 3 into relative position information with respect to the specific part captured in the optical image Q based on the position information of the ultrasound probe 1 acquired by the position sensor 3 and the optical image Q acquired by the optical camera 52. The relative position information conversion unit 54 can store a plurality of specific parts of the human body, such as a wrist, in advance as a reference part, detect one reference part and the ultrasound probe 1 captured in the optical image Q acquired by the optical camera 52, and convert the position information of the ultrasound probe 1 into the relative position information based on a positional relationship between the detected one reference part and the detected ultrasound probe 1 and the position information of the ultrasound probe 1.

The relative position information conversion unit 54 can detect the specific part and the ultrasound probe 1 from the optical image Q by, for example, a method of template matching, a method of using a trained model that has been trained in advance, through so-called machine learning, using a large number of optical images Q in which the specific part is captured and a large number of optical images Q in which the ultrasound probe 1 is captured, or the like. In addition, the relative position information conversion unit 54 can convert the position information of the ultrasound probe 1 into the relative position information by using, for example, a trained model that has been trained using a relationship between the positional relationship between the specific part of the human body and the ultrasound probe 1 in the optical image Q and the position information of the ultrasound probe 1 in the three-dimensional space.

The three-dimensional image data generation unit 24 generates three-dimensional ultrasound image data of the subject by using the relative position information converted by the relative position information conversion unit 54 as the position information of the ultrasound probe 1.

The centerline acquisition unit 25 acquires the centerline C of the blood vessel A from the three-dimensional ultrasound image data generated in this way, and the meandering degree calculation unit 26 calculates the meandering degree of the centerline C.

The guide unit 27 specifies the blood vessel region R suitable for the insertion of the insertion object based on the meandering degree, and guides the ultrasound probe 1 to the specified blood vessel region R based on the relative position information of the ultrasound probe 1.

As described above, with the ultrasound diagnostic apparatus of Embodiment 3, the relative position information conversion unit 54 converts the position information of the ultrasound probe 1 into the relative position information of the ultrasound probe 1 with respect to the position of the specific part of the human body captured in the optical image Q, and the guide unit 27 guides the ultrasound probe 1 to the blood vessel region R suitable for the insertion of the insertion object based on the meandering degree and the relative position information, so that the ultrasound probe 1 can be guided to the blood vessel region R with high accuracy even in a case where the posture of the subject is changed midway.

It has been described that the relative position information conversion unit 54, which is a feature of Embodiment 3, can be added to the ultrasound diagnostic apparatus of Embodiment 1, but the relative position information conversion unit 54 can also be added to the ultrasound diagnostic apparatus of Embodiment 2, which comprises the position sensor 53 configured by the marker detection unit 51 and the optical camera 52 instead of the position sensor 3.

### Embodiment 4

In Embodiments 1 to 3, the aspect in which only one blood vessel A is captured in the ultrasound images U1 and U2 has been described, but in some cases, a plurality of blood vessels A may be captured in the ultrasound images U1 and U2 depending on an observation site.

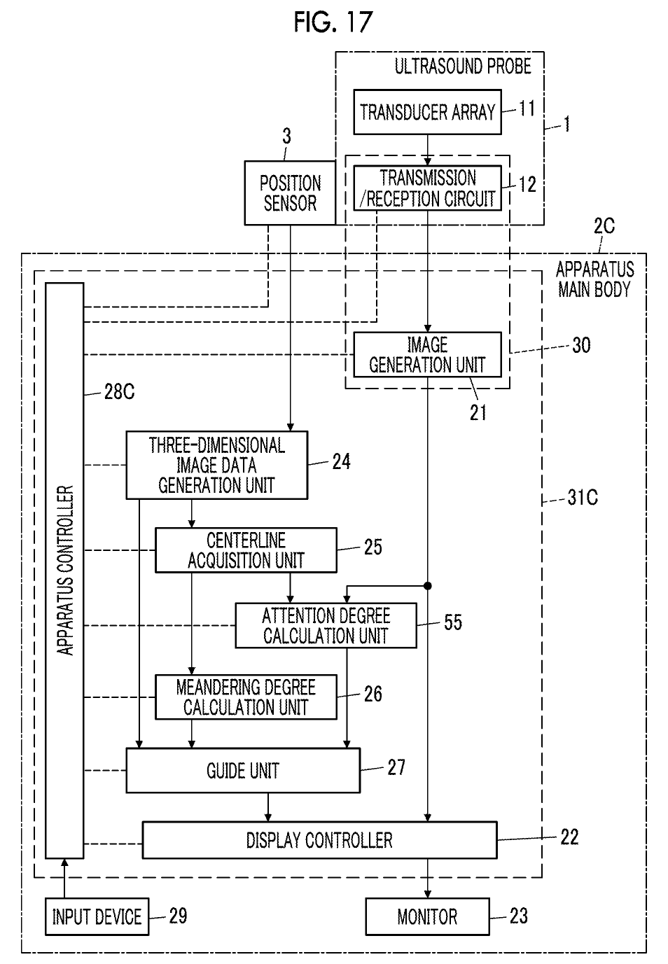

FIG. 17 shows a configuration of an ultrasound diagnostic apparatus of Embodiment 4. The ultrasound diagnostic apparatus of Embodiment 4 differs from the ultrasound diagnostic apparatus of Embodiment 1 shown in FIG. 1 in that an apparatus main body 2C is provided instead of the apparatus main body 2. The apparatus main body 2C in Embodiment 4 differs from the apparatus main body 2 in Embodiment 1 in that an attention degree calculation unit 55 is added and an apparatus controller 28C is provided instead of the apparatus controller 28.

In the apparatus main body 2C, the attention degree calculation unit 55 is connected to the image generation unit 21 and the centerline acquisition unit 25. The attention degree calculation unit 55 is connected to the guide unit 27 and the apparatus controller 28C. In addition, the image generation unit 21, the display controller 22, the three-dimensional image data generation unit 24, the centerline acquisition unit 25, the meandering degree calculation unit 26, the guide unit 27, the apparatus controller 28C, and the attention degree calculation unit 55 constitute a processor 31C for the apparatus main body 2C.

The image acquisition unit 30 acquires the plurality of frames of ultrasound images U1 representing the short-axis images of the plurality of blood vessels A.

The three-dimensional image data generation unit 24 generates three-dimensional ultrasound image data of the inside of the subject including the three-dimensional structures of the plurality of blood vessels A based on the plurality of frames of ultrasound images U1 acquired by the image acquisition unit 30 and the position information of the ultrasound probe 1 acquired by the position sensor 3.

The centerline acquisition unit 25 acquires a centerline C of each of the plurality of blood vessels A based on the three-dimensional ultrasound image data generated by the three-dimensional image data generation unit 24.

The attention degree calculation unit 55 calculates an attention degree of each of the plurality of blood vessels A based on the positions of the plurality of blood vessels A in each of the plurality of frames of ultrasound images U1 acquired by the image acquisition unit 30 or the length of the centerline C acquired by the centerline acquisition unit 25 for each of the plurality of blood vessels A. Here, the attention degree is an indicator representing a degree to which the user pays attention to each of the plurality of blood vessels A.

For example, it can be determined that the closer the position of the blood vessel A in the ultrasound image U1 is to the center of the ultrasound image U1, the higher the attention degree from the user. Therefore, the attention degree calculation unit 55 can calculate, for example, an average position of each of the plurality of blood vessels A in the plurality of frames of ultrasound images U1, and can assign a higher attention degree to the blood vessel A as the average position is closer to the center of the ultrasound image U1.

In addition, since the user usually moves the ultrasound probe 1 along the blood vessel A of interest on the body surface BS of the subject to acquire the plurality of frames of ultrasound images U1, it can be determined, for example, that the longer the centerline C of the blood vessel A is in the traveling direction D2 of the blood vessel A, the higher the attention degree from the user. Therefore, the attention degree calculation unit 55 can assign a higher attention degree to the blood vessel A having a longer centerline C in the traveling direction D2, for example.

The guide unit 27 designates the blood vessel A having the largest attention degree among the plurality of attention degrees calculated for the plurality of blood vessels A by the attention degree calculation unit 55, as the blood vessel A to be guided. The guide unit 27 further guides the ultrasound probe 1 to the designated blood vessel A based on the meandering degree calculated by the meandering degree calculation unit 26.

As described above, with the ultrasound diagnostic apparatus of Embodiment 4, the attention degree calculation unit 55 calculates the attention degree of each of the plurality of blood vessels A captured in the ultrasound image U1, and the guide unit 27 guides the ultrasound probe 1 to the blood vessel A having the largest attention degree, so that the ultrasound images U1 and U2 of the blood vessel region R suitable for the insertion of the insertion object can be acquired even in a case where the plurality of blood vessels A are captured in the ultrasound image U1.

As the configuration of the ultrasonic diagnostic apparatus according to Embodiment 4, a configuration has been described in which the attention degree calculation unit 55 is added to the ultrasonic diagnostic apparatus according to Embodiment 1, but the attention degree calculation unit 55 can also be added to the ultrasonic diagnostic apparatuses according to Embodiments 2 and 3.

### Embodiment 5

In Embodiment 4, it has been described that, in a case where the plurality of blood vessels A are captured in the ultrasound image U1, the blood vessel A to be guided is designated based on the attention degree, but, for example, it is also possible to calculate a suitability degree of each of the plurality of blood vessels A as a target for inserting the insertion object, and designate the blood vessel A to be guided based on the calculated suitability degree.

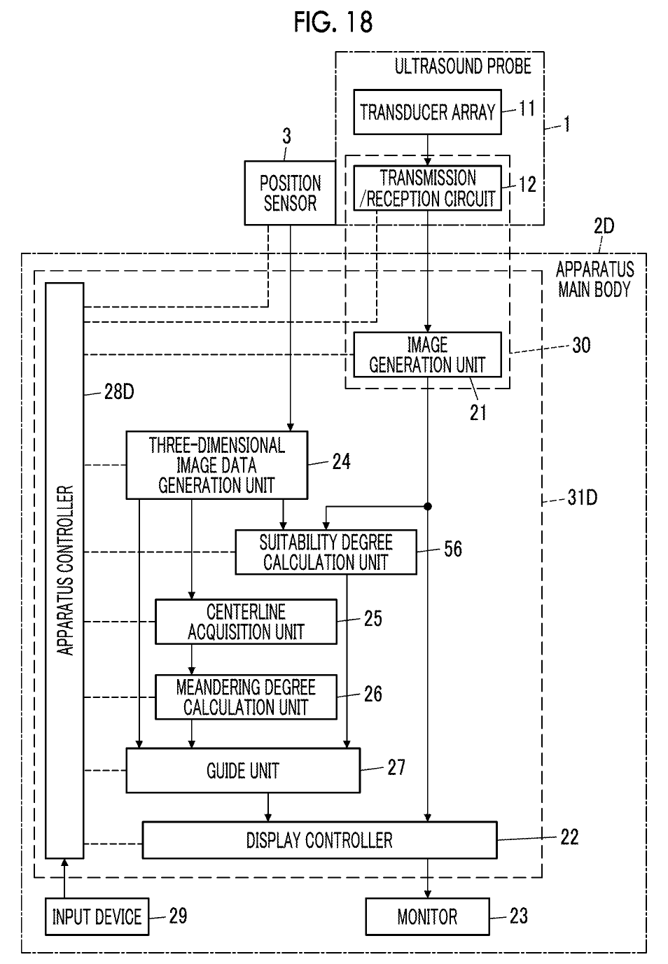

FIG. 18 shows a configuration of an ultrasound diagnostic apparatus of Embodiment 5. The ultrasound diagnostic apparatus of Embodiment 5 differs from the ultrasound diagnostic apparatus of Embodiment 1 shown in FIG. 1 in that an apparatus main body 2D is provided instead of the apparatus main body 2. The apparatus main body 2D in Embodiment 5 differs from the apparatus main body 2 in Embodiment 1 in that a suitability degree calculation unit 56 is added and an apparatus controller 28D is provided instead of the apparatus controller 28.

In the apparatus main body 2D, the suitability degree calculation unit 56 is connected to the image generation unit 21 and the three-dimensional image data generation unit 24. The suitability degree calculation unit 56 is connected to the guide unit 27 and the apparatus controller 28D. In addition, the image generation unit 21, the display controller 22, the three-dimensional image data generation unit 24, the centerline acquisition unit 25, the meandering degree calculation unit 26, the guide unit 27, the apparatus controller 28D, and the suitability degree calculation unit 56 constitute a processor 31D for the apparatus main body 2D.

The suitability degree calculation unit 56 calculates a suitability degree of each of the plurality of blood vessels A based on the depth of each of the plurality of blood vessels A with respect to the body surface BS of the subject or the inner diameter of each of the plurality of blood vessels A by referring to the three-dimensional ultrasound image data including the three-dimensional structures of the plurality of blood vessels A generated by the three-dimensional image data generation unit 24. Here, the suitability degree is an indicator representing a degree to which the blood vessel A is suitable for the insertion of the insertion object.

For example, in general, the shallower the position of the blood vessel A is, the easier it is to insert the insertion object into the blood vessel A, and hence the blood vessel A can be determined to have a higher insertion suitability degree. Therefore, the suitability degree calculation unit 56 can calculate, for example, an average depth value of each of the plurality of blood vessels A over the entire corresponding centerline C, and can assign a higher suitability degree to the blood vessel A having a smaller calculated average depth value.

In addition, in a case where the insertion object is a catheter, in a case where an inner diameter of the blood vessel A is smaller than an outer diameter of the catheter, it is impossible to insert the catheter into the blood vessel A, and, in a case where the inner diameter of the blood vessel A is too large compared to the outer diameter of the catheter, the catheter may not be able to sufficiently expand the blood vessel A, resulting in a reduced treatment effect. Therefore, the suitability degree calculation unit 56 can calculate, for example, an average inner diameter value of each of the plurality of blood vessel A over the entire corresponding centerline C, and can assign a higher suitability degree to the blood vessel A whose calculated average inner diameter value is closer to the predetermined recommended inner diameter value. The recommended inner diameter value can be set to, for example, about three times the outer diameter of the catheter.

The guide unit 27 designates the blood vessel A having the largest suitability degree among the plurality of suitability degrees calculated for the plurality of blood vessels A by the suitability degree calculation unit 56, as the blood vessel A to be guided. The guide unit 27 further guides the ultrasound probe 1 to the designated blood vessel A based on the meandering degree calculated by the meandering degree calculation unit 26.

As described above, with the ultrasound diagnostic apparatus of Embodiment 5, the suitability degree calculation unit 56 calculates the suitability degree of each of the plurality of blood vessels A captured in the ultrasound image U1, and the guide unit 27 guides the ultrasound probe 1 to the blood vessel A having the largest suitability degree, so that the ultrasound images U1 and U2 of the blood vessel region R suitable for the insertion of the insertion object can be acquired even in a case where the plurality of blood vessels A are captured in the ultrasound image U1.

As the configuration of the ultrasonic diagnostic apparatus according to Embodiment 5, a configuration has been described in which the suitability degree calculation unit 56 is added to the ultrasonic diagnostic apparatus according to Embodiment 1, but the suitability degree calculation unit 56 can also be added to the ultrasonic diagnostic apparatuses according to Embodiments 2 and 3.

### EXPLANATION OF REFERENCES

1, 1A: ultrasound probe

2, 2A, 2B, 2C, 2D: apparatus main body

3, 53: position sensor

11: transducer array

12: transmission/reception circuit

21: image generation unit

22: display controller

23: monitor

24: three-dimensional image data generation unit

25: centerline acquisition unit

26: meandering degree calculation unit

27: guide unit

28, 28A, 28B, 28C, 28D: apparatus controller

29: input device

30: image acquisition unit

31, 31A, 31B, 31C, 31D: processor

41: pulser

42: amplification unit

43: AD conversion unit

44: beam former

45: signal processing unit

46: DSC

47: image processing unit

51: marker detection unit

52: optical camera

54: relative position information conversion unit

55: attention degree calculation unit

56: suitability degree calculation unit

A: blood vessel

B: marker

BS: body surface

C: centerline

D1: transverse diameter direction

D2: traveling direction

E: average line

F: arrow

G1, G2, G3, G4: section

H: hand

J1, J2: inflection point

K1: interval

L1, L2: distance

M: arm

Q: optical image

R: blood vessel region

SP: scanning plane

U1, U2: ultrasound image

What is claimed is:

1. An ultrasound diagnostic apparatus comprising:
an ultrasound probe;
a position sensor that acquires position information of the ultrasound probe; and
a processor configured to:
acquire a plurality of frames of ultrasound images obtained by capturing a blood vessel of a subject by transmitting and receiving ultrasound beams using the ultrasound probe;
generate three-dimensional ultrasound image data of the subject based on the position information of the ultrasound probe and the plurality of frames of ultrasound images and representing a short-axis image of the blood vessel;
acquire a centerline of the blood vessel in a three-dimensional space based on the three-dimensional ultrasound image data;
calculate a meandering degree of the centerline in a transverse diameter direction of the blood vessel, the transverse diameter direction being perpendicular to a plane corresponding to the short-axis image of the blood vessel; and
guide the ultrasound probe to a range on the centerline based on the meandering degree.

2. The ultrasound diagnostic apparatus according to claim 1,
wherein the processor is configured to;
divide the centerline into a plurality of sections having a predetermined length; and
calculate the meandering degree in each of the plurality of sections.

3. The ultrasound diagnostic apparatus according to claim 2,
wherein the processor is configured to:
calculate an average position of the centerline in the transverse diameter direction; and
calculate, in each of the plurality of sections, the number of inflection points of the centerline whose distance from the average position is equal to or greater than a predetermined position threshold value, as the meandering degree.

4. The ultrasound diagnostic apparatus according to claim 2,
wherein the processor is configured to calculate, in the plurality of sections, a reciprocal of an interval between adjacent inflection points of the centerline, as the meandering degree.

5. The ultrasound diagnostic apparatus according to claim 1,
wherein the processor is configured to guide the ultrasound probe to a range on the centerline where the meandering degree is equal to or less than a predetermined meandering degree threshold value.

6. The ultrasound diagnostic apparatus according to claim 2,
wherein the processor is configured to guide the guide unit guides the ultrasound probe to a range on the centerline where the meandering degree calculated by the meandering degree calculation unit is equal to or less than a predetermined meandering degree threshold value.

7. The ultrasound diagnostic apparatus according to claim 3,
wherein the processor is configured to guide the guide unit guides the ultrasound probe to a range on the centerline where the meandering degree calculated by the meandering degree calculation unit is equal to or less than a predetermined meandering degree threshold value.

8. The ultrasound diagnostic apparatus according to claim 4,
wherein the processor is configured to guide the guide unit guides the ultrasound probe to a range on the centerline where the meandering degree calculated by the meandering degree calculation unit is equal to or less than a predetermined meandering degree threshold value.

9. The ultrasound diagnostic apparatus according to claim 1,
wherein the processor is configured to:
acquire a depth of the blood vessel with respect to a body surface of the subject over an entire centerline by referring to the three-dimensional ultrasound image data; and
guide the ultrasound probe to a range where the meandering degree is equal to or less than a predetermined meandering degree threshold value and the depth of the blood vessel is equal to or less than a predetermined depth threshold value.

10. The ultrasound diagnostic apparatus according to claim 2,
wherein the processor is configured to:
acquire a depth of the blood vessel with respect to a body surface of the subject over an entire centerline by referring to the three-dimensional ultrasound image data; and
guide the ultrasound probe to a range where the meandering degree is equal to or less than a predetermined meandering degree threshold value and the depth of the blood vessel is equal to or less than a predetermined depth threshold value.

11. The ultrasound diagnostic apparatus according to claim 3,
wherein the processor is configured to:
acquire a depth of the blood vessel with respect to a body surface of the subject over an entire centerline by referring to the three-dimensional ultrasound image data; and
guide the ultrasound probe to a range where the meandering degree is equal to or less than a predetermined meandering degree threshold value and the depth of the blood vessel is equal to or less than a predetermined depth threshold value.

12. The ultrasound diagnostic apparatus according to claim 4,
wherein the processor is configured to:
acquire a depth of the blood vessel with respect to a body surface of the subject over an entire centerline by referring to the three-dimensional ultrasound image data; and
guide the ultrasound probe to a range where the meandering degree is equal to or less than a predetermined meandering degree threshold value and the depth of the blood vessel is equal to or less than a predetermined depth threshold value.

13. The ultrasound diagnostic apparatus according to claim 1,
wherein the processor is configured to:
acquire an inner diameter of the blood vessel over an entire centerline by referring to the three-dimensional ultrasound image data; and
guide the ultrasound probe to a range where the meandering degree is equal to or less than a predetermined meandering degree threshold value and the inner diameter of the blood vessel is closest to a predetermined recommended inner diameter value.

14. The ultrasound diagnostic apparatus according to claim 2,
wherein the meandering degree is equal to or less than a predetermined meandering degree threshold value in two or more of the plurality of sections, and
the processor is configured to guide the ultrasound probe to a section having a smallest meandering degree among the two or more sections.

15. The ultrasound diagnostic apparatus according to claim 1, further comprising:
a monitor,
wherein the processor is configured to display a guide for the ultrasound probe on the monitor.

16. The ultrasound diagnostic apparatus according to claim 15,
wherein a marker is disposed on the ultrasound probe,
the position sensor includes an optical camera configured to acquire an optical image in which the ultrasound probe is captured,
the position sensor is configured to acquire the position information of the ultrasound probe by detecting the marker captured in the optical image acquired by the optical camera, and
the processor is configured to display, on the monitor, the guide for the ultrasound probe by superimposing the guide for the ultrasound probe on the optical image, based on the position information of the ultrasound probe acquired by the position sensor.

17. The ultrasound diagnostic apparatus according to claim 1, further comprising:
an optical camera configured to acquire an optical image in which the ultrasound probe and a specific part of the subject are captured, and
wherein the processor is configured to:
convert, based on the position information acquired by the position sensor and the optical image acquired by the optical camera, the position information into relative position information with respect to the specific part captured in the optical image; and
use the relative position information as the position information of the ultrasound probe acquired by the position sensor.

18. The ultrasound diagnostic apparatus according to claim 1,
wherein a plurality of the blood vessels are captured in each of the plurality of frames of ultrasound images, and
the processor is configured to:
calculate an attention degree of each of the plurality of blood vessels based on positions of the plurality of blood vessels in each of the plurality of frames of ultrasound images or a length of the centerline for each of the plurality of blood vessels; and
guide the ultrasound probe on a blood vessel having a largest attention degree among a plurality of the attention degrees, based on the meandering degree.

19. The ultrasound diagnostic apparatus according to claim 1,
wherein a plurality of the blood vessels are captured in each of the plurality of frames of ultrasound images, and
the processor is configured to:
calculate a suitability degree of each of the plurality of blood vessels based on a depth of the blood vessel with respect to a body surface of the subject or an inner diameter of the blood vessel by referring to the three-dimensional ultrasound image data, and
guide the ultrasound probe on a blood vessel having a largest suitability degree among a plurality of the suitability degrees, based on the meandering degree.

20. A control method of an ultrasound diagnostic apparatus, the control method comprising:
acquiring position information of an ultrasound probe;
acquiring a plurality of frames of ultrasound images that are obtained by capturing a blood vessel of a subject by transmitting and receiving ultrasound beams using the ultrasound probe and that represent a short-axis image of the blood vessel;
generating three-dimensional ultrasound image data of the subject based on the position information of the ultrasound probe and the plurality of frames of ultrasound images;
acquiring a centerline of the blood vessel in a three-dimensional space based on the three-dimensional ultrasound image data;
calculating a meandering degree of the centerline in a transverse diameter direction of the blood vessel, the transverse diameter direction being perpendicular to a plane corresponding to the short-axis image of the blood vessel; and
guiding the ultrasound probe to a range on the centerline based on the meandering degree.
